SEQUENCE LISTING (1 sequence)

SEQ ID NO: 1 - protein, 37 aa, Artificial sequence
  collagen mimetic peptide
GKKGPXGPXGPXGPXGFXGERGPXGPXGPXGPXGQQG

(19) United States
(12) Patent Application Publication
(10) Pub. No.: US 2016/0194378 A1
(43) Pub. Date: Jul. 7, 2016

(54) PEPTIDES AND USES THEREOF

(71) Applicant: HEART BIOTECH LIMITED, London (GB)

(72) Inventors: Anthony Edward George CASS, Hertfordshire (GB); Magdi Habib YACOUB, London (GB)

(21) Appl. No.: 14/778,151

(22) PCT Filed: Mar. 18, 2014
(86) PCT No.: PCT/GB2014/050844

§ 371 Date: Sep. 18, 2015

(30) Foreign Application Priority Data
Mar. 18, 2013 (GB) 1304947.3

(51) Int. Cl. C07K 14/78; A61L 27/24

(52) U.S. Cl. CPC C07K 14/78; A61L 27/24; A61K 38/00

(57) ABSTRACT

Provided is a peptide and a hydrogel prepared therefrom. The peptide comprises the sequence Gly-(A)-(Gly-X-Y)ₘ-M-(Gly-X-Y)ₙ-Gly-(B)-Gly, wherein A and B are telopeptides, X and Y are any amino acid, m and n are integers of at least 3 and M is a receptor binding sequence. Artificial collagen derived from the peptide of the invention provides an alternative to the use of animal- or human-derived collagen in industrial, biological and biomedical applications.

The present invention relates to artificial peptides. In particular, the present invention relates to artificial peptides and their use in the preparation of a hydrogel which mimics natural collagen.

### BACKGROUND TO THE INVENTION

Collagen is the most abundant structural protein in mammals and is the main component of the natural extracellular matrix (ECM). Collagen builds tissue-specific architecture and mediates cell attachment, morphology, proliferation and migration. It provides mechanical strength and structural integrity to various tissues including the skin, tendons, bones, blood vessels, cartilage, ligament and teeth, and occurs as fibrous inclusions in most other body structures. To date, 28 different types of collagen have been identified as being involved in shaping and maintaining the ECM by forming fibrillar or other large-scale assemblies.

Collagen in its native form is typically a rigid, rod-shaped molecule approximately 300 nm long and 1.5 nm in diameter. The collagen triple helical conformation is comprised of three left handed polyproline II (PPM-like helical chains of length about ~1050 amino acids, wound around each other to form a tightly packed right-handed superhelix. The amino acid sequence of the collagen primary structure revealed the presence of a triple helix-forming middle section flanked between non-helical telopeptides on both ends. The triple helical region is composed of mostly Gly-X-Y triplets, wherein X and Y are often proline and hydroxyproline respectively. The length of the non-helical telopeptide regions constitutes less than about 5% of the collagen molecule and is responsible for the Lysyl Oxidase (LO) mediated cross-linking between collagen nanofibers which leads the formation of hydrogel.

Collagen is a very popular biomaterial due to its excellent biocompatibility. Collagen-based biomaterials for tissue engineering and medical products have been approved by FDA and are commercially available. These include collagen-based corneal shields, hemostatic sponges, wound dressings and blood vessel replacements. In addition, collagen in the sub-cutaneous fat contributes to the shape and contouring of different areas of the body. It is widely used for this purpose and in a variety of anti-ageing preparations by the cosmetics industry.

Typically, clinical grade commercial collagens are extracted from a mammalian source, decellularized, purified and sterilized to the extent feasible without denaturing the molecule, and often chemically modified for specialized use. While natural collagen has many advantages including biocompatibility, shapability, and haemostatic properties, the use of mammal (e.g. bovine- or porcine-) derived collagen presents potential hazards, especially the transmission of hidden diseases and pathogens. Animal collagen may also be culturally unacceptable. The use of collagen of human origin has not obviated problems of possible contagion.

Efforts have been made to replicate natural collagen using Collagen Mimetic Peptides (CMPs). However, attempts to make artificial collagen using CMPs have encountered numerous problems including the inability of CMPs to form stable hydrogels, a lack of integrin binding sites or the presence of integrin binding sites inhibiting the formation of higher order structures, and a lack of suitability for production on a commercial scale.

The present invention seeks to mitigate at least some of the problems identified above.

### SUMMARY OF THE INVENTION

According to a first aspect of the present invention, there is provided a peptide comprising the sequence Gly-(A)-(Gly-X-Y)ₘ-M-(Gly-X-Y)ₙ-Gly-(B)-Gly

wherein:

A and B are telopeptides;

X and Y are any amino acid;

m and n are integers of at least 3; and

M is an receptor binding sequence.

The synthetic peptide of the invention contains a triple helix-forming motif and non-helical telopeptide regions for enzymatic cross-linking. The peptide of the invention may thus be considered to be a collagen mimetic peptide since it has similar properties to the peptide which forms natural collagen. Like native collagen, the peptide of the present invention is biocompatible but is of a simplified oligopeptide structure which can be easily synthesized in the laboratory or on a commercial scale. The peptide of the invention can incorporate any desired cell-binding motif, and is free from potentially dangerous contaminants. Artificial collagen derived from the peptides of the invention provides an alternative to the use of animal- or human-derived collagen in industrial, biological and biomedical applications.

According to a second aspect of the present invention, there is provided a method for preparing a hydrogel, the method comprising:
providing a solution of the peptide according to the first aspect of the invention;
allowing the peptides to assemble into higher order structures; and
cross-linking the peptides.

The method of the invention provides an in vitro process for the preparation of an artificial collagen hydrogel which mimics the formation of natural collagen in vivo, and can be implemented without the use of any toxic chemicals. The method of the invention enables the preparation of artificial collagen which is free from the contaminants frequently associated with purified native collagen products, particularly mammalian pathogens.

According to a third aspect of the invention, there is provided a hydrogel comprising the peptide according to the first aspect of the invention.

According to a fourth aspect of the invention, there is provided a hydrogel obtainable by the method according to the second aspect of the invention.

A hydrogel prepared from the collagen mimetic peptide of the present invention may be considered to be an artificial or ‘biomimetic’ collagen since its structural and physical characteristics are similar to those of natural collagen. Since the peptide of the invention contains all of the key structural features of natural collagen, it is able to self-assemble into ordered triple helices.

By virtue of its biocompatibility, the artificial collagen (i.e. hydrogel) finds use in numerous applications, including therapeutic applications (e.g. grafts and implants) and cosmetics (e.g. collagen injections).

According to a fifth aspect of the invention, there is provided a material or composition comprising the hydrogel of the third or fourth aspect of the invention.

According to a sixth aspect of the invention, there is provided an article made from or comprising the material or composition according to the fifth aspect of the invention.

According to a seventh aspect of the invention, there is provided the use of the hydrogel of the third or fourth aspect of the invention in the generation of artificial tissue.

According to an eighth aspect of the invention, there is provided an artificial tissue comprising the hydrogel of the third or fourth aspect of the invention.

According to a ninth aspect of the invention, there is provided the hydrogel of the third or fourth aspect, or the artificial tissue of the seventh aspect, for use in therapy.

According to a tenth aspect of the invention, there is provided the hydrogel of the third or fourth aspect, or the artificial tissue of the seventh aspect, for use in regenerative therapy.

According to an eleventh aspect of the invention, there is provided a method of treating diseased or damaged tissue, the method comprising administering the hydrogel of the third or fourth aspect, or the artificial tissue of the seventh aspect, to a patient in need thereof.

### DETAILED DESCRIPTION OF THE INVENTION

The investigation that lead to the work described here began with the notion that a better synthetic route for the preparation of artificial collagen from simple collagen mimetic peptides might be achieved by following the processes adopted by natural collagen in vivo. The real challenge in the preparation of synthetic collagen is not only in generating the characteristic triple helices in solution, but also in recreating the three-dimensional structures which result in a cell-compatible hydrogel.

Various attempts to prepare artificial collagen using synthetic CMPs have been documented. However, all available literature reports the modification of the triple helical region of the peptide sequence to induce stimuli-responsive self-assembly formation. There are no reports of either replicating the native collagen architecture or synthetic methods based on the process adopted by natural collagen for hydrogel formation. The hypothesis underlying the synthesis of the peptides and hydrogel of the invention was therefore to design a collagen mimetic peptide with the same structural architecture as natural collagen.

The collagen mimetic peptide of the invention comprises the sequence

Gly-(A)-(Gly-X-Y)ₘ-M-(Gly-X-Y)ₙ-Gly-(B)-Gly

wherein:

A and B are telopeptides, each comprising a residue that is capable of being cross-linked;

X and Y are any amino acid;

m and n each are integers of at least 3; and

M comprises a receptor binding sequence.

The Gly-X-Y motif promotes the formation of triple helices, as found in natural collagen. X and Y may be the same amino acid, or they may be different. In some embodiments, X and/or Y is selected from proline and hydroxyproline. For example, X may be proline or hydroxyproline, while Y is any amino acid. In another example, Y may be proline or hydroxyproline, while X is any amino acid. In some embodiments, both X and Y are proline. In other embodiments, both X and Y are hydroxyproline. Alternatively, one of X and Y may be proline, and the other may be hydroxyproline. In some embodiments, X is proline and Y is hydroxyproline.

It will be appreciated that the number of repeats of the Gly-X-Y motifs must be sufficient to enable helix formation. It is generally considered that at least three repeats are required for stable helix formation. Thus, in some embodiments, the value of each of m and n is at least 3, at least 4 or at least 6. Any number of repeats may be included, although the cost of peptide synthesis may limit the length of the peptide chain in practice. In some embodiments, m and n are integers of no more than 100, no more than 50, no more than 20 or no more than 10. In further embodiments, m and n are integers of from 3 to 6. The values of m and n may be the same, or they may be different.

As used herein, “telopeptide” will be understood to be a generic term for a sequence of amino acids which does not itself form a triple helix. Thus, A and B are sequences which do not form helices. The telopeptide regions advantageously provide sites for enzymatic cross-linking, as in natural collagen.

In some embodiments, each of A and B is a sequence of two or more amino acids, wherein the sequence comprises at least one lysine or glutamine residue.

The sequences of A and B may contain the same number of amino acids, or they may be different in length. In some embodiments, the sequence of A and/or B is at least 2, at least 3, at least 4 or at least 5 amino acids in length. In some embodiments, the sequence of A and/or B is no more than 100, no more than 50, no more than 20, no more than 10 or no more than 5 amino acids in length. In further embodiments, the sequence of A and/or B is from 2 to 5 amino acids in length.

In some embodiments, the sequence of A and/or B comprises or consists of two or more lysine and/or glutamine residues.

In some embodiments, A comprises the sequence Lys-Lys (KK). In some further embodiments, A is constituted by the sequence Lys-Lys (KK).

In some embodiments, B comprises the sequence Gln-Gln (QQ). In some embodiments, B is constituted by the sequence Gln-Gln (QQ).

M comprises a receptor binding sequence. Any suitable receptor sequence could be used, although it will be understood that the length of the receptor binding sequence should be sufficiently short so as not to disrupt formation of the triple helix. In some embodiments, the receptor binding sequence is no more than 10, no more than 8 or no more than 6 amino acids in length.

In some embodiments, M comprises an integrin binding sequence. As will be known to those skilled in the art, integrins are transmembrane receptors that mediate attachment of cells to other cells or to the ECM. Different proteins of the ECM are recognized by different integrins. In particular, integrins can mediate the attachment of cells to the collagen of the ECM. In humans, collagen binding is primarily provided by integrins α1β1, α2β1, α10β1 a α11β1. As used herein, an “integrin binding sequence” will be understood to mean a sequence of amino acids which is capable of interacting with cell receptors.

M may comprise any integrin binding sequence found in natural collagen, or any synthetic sequence which is capable of binding integrins. In some embodiments, M comprises or consists of the sequence GFOGER, GFOGDR or GFOLDV (based on the one-letter code for amino acids, wherein O is hydroxyproline). In some particular embodiments, M comprises or consists of the sequence GFOGER.

In some embodiments, the peptide comprises or consists of the following sequence:

Gly- Lys-Lys-(Gly-X-Y)ₘ-M-(Gly-X-Y)ₙ-Gly-Glu-Glu-
Gly

wherein:

X and Y are proline and hydroxyproline, respectively;

m and n are integers of at least 3, for example from 4 to 6 and

M comprises an integrin binding sequence.

A hydrogel may be prepared from the peptides of the invention using the following method:
providing a solution of the peptides according to the first aspect of the invention;
allowing the peptides to assemble into higher order structures; and
cross-linking the peptides.

The solution may comprise the peptides in any suitable buffer, e.g. TRIS (tris(hydroxymethyl)aminomethane).

The concentration of peptide in the solution must be sufficient to provide a hydrogel. It will be appreciated that the peptide concentration required may depend on a number of factors, such as the length of the peptide. In some embodiments, the concentration of the peptide solution is at least 5% or at least 8% (w/v). The solution may be prepared by adding the peptide to a buffer up to the limit of solubility, i.e. to provide a saturated solution. Alternatively, the peptide concentration may be no more than 30% or no more than 20%. In some embodiments, the concentration of peptide in the solution is from 8% to 12%, e.g. 10%.

The peptides of the invention can be prepared using standard peptide synthesis techniques. Alternatively, the peptides may be produced using recombinant technology, e.g. by expressing a DNA sequence encoding the peptide in a microorganism. The design of a nucleic acid sequence which encodes a desired peptide and methods for the expression of that nucleic acid sequence using genetic engineering of microorganisms are techniques commonly known to those skilled in the art.

The peptides of the invention will spontaneously assemble in solution to form triple helices by virtue of their Gly-X-Y motifs. The triple helices may assemble further into longer, fatter, structures known as fibrils.

The step of allowing the peptides to form higher order structures may thus comprise incubating the solution for a period of time sufficient for triple helix and fibril formation to occur. In some embodiments, the step of allowing the peptides to assemble into higher order structures comprises incubating the solution for at least 30 seconds, at least 1 minute, at least 5 minutes, at least 10 minutes, at least 30 minutes or at least 1 hour. In some embodiments, the solution is incubated overnight. The solution may be incubated at a temperature of from 4° C. to 37° C.

The step of cross-linking the peptides results in the formation of covalent bonds both between the peptides within a triple helix and also between different triple helices and different fibrils, thereby forming a network. More specifically, cross-links are formed between the telopeptide (‘A’ and ‘B’) regions of the peptide.

Cross-linking may be effected by adding an enzyme to the peptide solution. The amount of enzyme required to effect cross-linking can be determined empirically by those skilled in the art. If too little enzyme is used the gel formation will be too slow to be practical. Too much enzyme will increase the cost of gel formation without providing any advantage, and may also unnecessarily contaminate the hydrogel.

The enzyme may be lysyl oxidase (LOX), which is involved in the formation of natural collagen. Lysyl oxidase catalyses the formation of aldehydes from the lysine residues in the peptide. These aldehydes react with each other or with unmodified lysine residues, forming covalent bonds. Thus, if cross-linking is to be carried out using lysyl oxidase, both of the telopeptides ‘A’ and ‘B’ must comprise a lysine residue.

In some embodiments, the enzyme is transglutaminase. Transglutaminase catalyses the formation of a covalent bond between a free amine group and the acyl group of glutamine. Thus, if cross-linking is to be carried out using transglutaminase, the telopeptide ‘A’ must comprise a lysine residue while the telopeptide ‘B’ must comprise a glutamine residue. The use of transglutaminase to effect cross-linking is advantageous since this enzyme is commercially available.

It will be appreciated that if cross-linking is carried out enzymatically, the buffer should be chosen so as to not inhibit the enzyme. In some embodiments, the buffer is slightly alkaline and close to the pH optimum of the cross-linking enzyme. Since the cross-linking is carried out under mild conditions and in the absence of highly reactive chemical agents, it is possible to carry out cross-linking in the presence of cells. Thus, in some embodiments, the method further comprises adding cells to the higher order structures formed from the peptides in solution, and effecting cross-linking in the presence of the cells. This enables the entrapment of cells in the hydrogel.

In some embodiments, cross-linking is carried out in the presence of a reducing agent. A reducing agent conveniently increases the efficiency of cross-linking. Suitable reducing agents include glutathione, dithiothreitol (DTT), beta-mercaptoethanol and dihydrolipoic acid. Glutathione is particularly advantageous since it is naturally occurring in animals. As such, hydrogels produced using glutathione are fully biocompatible. The reducing agent may be used at a concentration of from 1 to 100 mM.

The method may further comprise incubating the reaction mixture, i.e. the solution comprising the peptide, the enzyme and, optionally, the reducing agent, for a period of time sufficient for hydrogel formation to occur. It will be appreciated that the length of time required will depend on a number of factors including the peptide concentration and the amount of enzyme present. The mixture may be incubated for at least 30 minutes, at least 1 hour, at least 3 hours, at least 12 hours or overnight. The mixture may be incubated at a temperature of from 4 to 37° C.

The present invention thus further provides a hydrogel comprising the peptide according to the first aspect of the invention. The hydrogel may be obtainable using the method of the second aspect of the invention.

In some embodiments, the peptide of the first aspect of the invention constitutes at least 30%, at least 40%, at least 50%, at least 60%, at least 70%, at least 80%, at least 90%, at least 95% or at least 98% of the hydrogel. In some embodiments, the hydrogel is entirely constituted by the peptide according to the first aspect of the invention.

In some embodiments, the hydrogel comprises the peptide of the first aspect of the invention and one or more other proteins. For example, peptides derived from fibrin or elastin could be co-gelled with the peptide of the invention in the preparation of a hydrogel.

In some embodiments, the hydrogel is thermoreversible. By “thermoreversible”, it will be understood that the hydrogel has the ability to gel reversibly when subjected to a change of temperature.

In some embodiments, the hydrogel has a thermal stability (i.e. melting temperature) which is higher than that of native (i.e. naturally occurring) collagen. In some embodiments, the thermal stability of the hydrogel is from 40 to 50° C., or from 42 to 47° C., e.g. about 45° C.

In some embodiments, the hydrogel is transparent. Transparency is particularly useful for certain medical applications, such as ophthalmic applications.

The invention further resides in a material or composition comprising the hydrogel of the third or fourth aspect of the invention.

Such a material may find use in the treatment of skin, for example wounds, burns, scars and bed sores. In some embodiments, the material forms the part or the whole of an article for application to damaged, diseased or infected skin.

Thus, the present invention also resides in an article made from or comprising the material according to the fifth aspect of the invention. In some embodiments, the article is a wound dressing, hemostatic sponge or other healing aid. In other embodiments, the article is a corneal shield.

The article may comprise two or more layers, at least one of which is formed from the material of the invention. For example, a wound dressing may comprise a wound-contacting layer comprising or consisting or the material of the invention, and one or more further layers (e.g. backing layers, adhesive layers).

The hydrogel of the third or fourth aspect of the invention may be used in the generation of artificial tissue.

For example, the hydrogel of the invention may be used to generate artificial tendons, organs, ligaments, corneas, cartilage, blood vessels, bone grafts and heart valves.

Artificial tissue may be prepared by molding the hydrogel of the present invention. Alternatively, the hydrogel could be electrospun or 3-D printed. Cells may be cultured on the hydrogel structure, or entrapped within the hydrogel structure, prior to implantation into a patient. Alternatively, a molded or shaped hydrogel structure may be implanted as an acellular construct.

Such artificial tissue may be used for the treatment of diseased or damaged tissue including skin (e.g. bed sores, hypertrophic scarring, burns), ligaments (e.g. ligament inflammation and rupture), tendons (e.g. inflammation, rupture), vessels (e.g. aneurisms, arteriosclerosis, atherosclerosis, vessel grafts), organ tissue (e.g. heart, liver, pancreas), valves (e.g. heart valves) or eyes (e.g. corneas) in a patient in need thereof. In particular, artificial tissue prepared using the hydrogel of the invention may find use in the treatment of cardiovascular disease, for example heart valve disease. The patient may be animal or human.

Thus, the present invention also resides in the hydrogel of the invention, or artificial tissue generated therefrom, for use in therapy.

The present invention further resides in the hydrogel of the invention, or artificial tissue generated therefrom, for use in regenerative therapy.

As used herein, “regenerative therapy” will be understood to mean facilitating the replacement and/or regeneration of human cells, tissues or organs or to aid or establish normal function. For example, the hydrogel of the invention may be applied onto or implanted into a human or animal body to facilitate the repair of damaged tissue, and/or to stimulate the growth of new tissue. Alternatively, the hydrogel of the invention may be used provide a scaffold for the growth of cells, tissues or organs outside of the body (i.e. artificial tissue engineering). The generation of new tissue may involve the use of stem cells or cells taken from the body of the patient to be treated. It will be appreciated that in vivo, the hydrogel degrades and is replaced by natural collagen. The biomimetic collagen of the invention thus acts as a transient substitute for normal collagen.

The present invention further provides a method of treating diseased or damaged tissue. The method may comprise administering the hydrogel of the invention to a patient in need thereof.

The hydrogel may be administered topically (e.g. by application to the skin). Alternatively, the hydrogel may be implanted into the body. The hydrogel may be used to replace existing tissues or it may be grafted onto existing tissues.

Embodiments of the invention will now be described by way of example with reference to the accompanying Figures, in which:

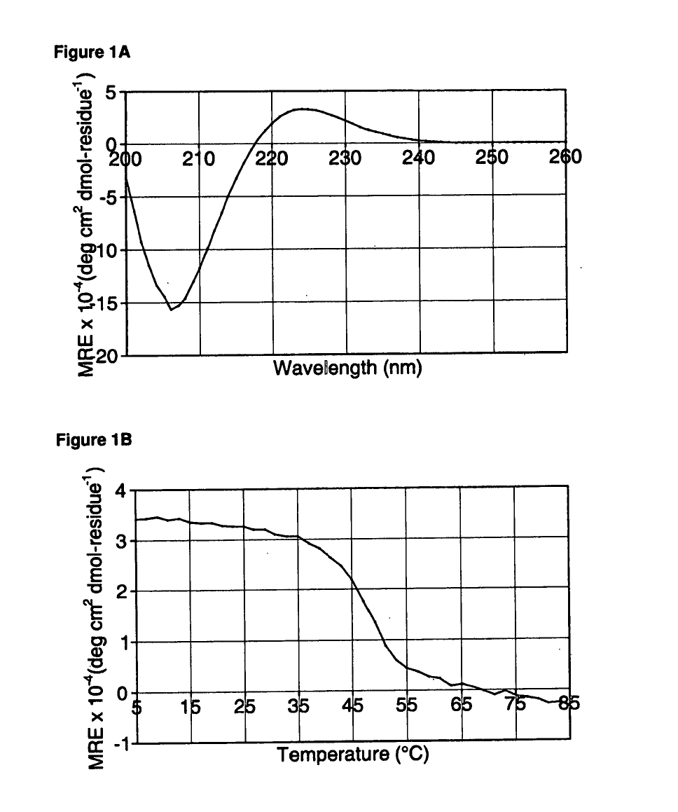

FIG. 1A is a Circular Dichroism (CD) spectrum of a collagen mimetic peptide in accordance of the present invention designated CMP-KQ (wherein ‘KO’ designates lysine-glutamine) in TRIS buffer at 15° C., showing the formation of a triple helical structure;

FIG. 1B is a CD spectrum of the CMP-KQ peptide in TRIS buffer, showing thermal unfolding. 0.5 mg/ml CMP-KQ was incubated at 4° C. overnight prior to measurement;

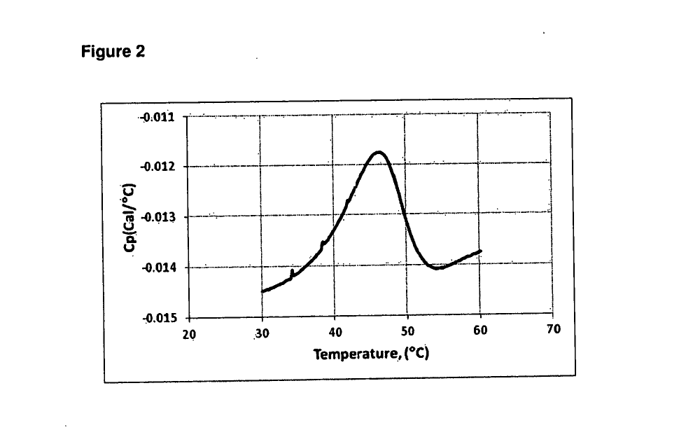

FIG. 2 is a differential scanning calorimetry thermogram of the CMP-KQ peptide in TRIS buffer. The concentration of CMP-KQ was 36 mg/ml and incubated at 4° C. overnight. The peptide and buffer solutions were degassed for 1 hour prior to measurements. The heating rate was 10° C./hr;

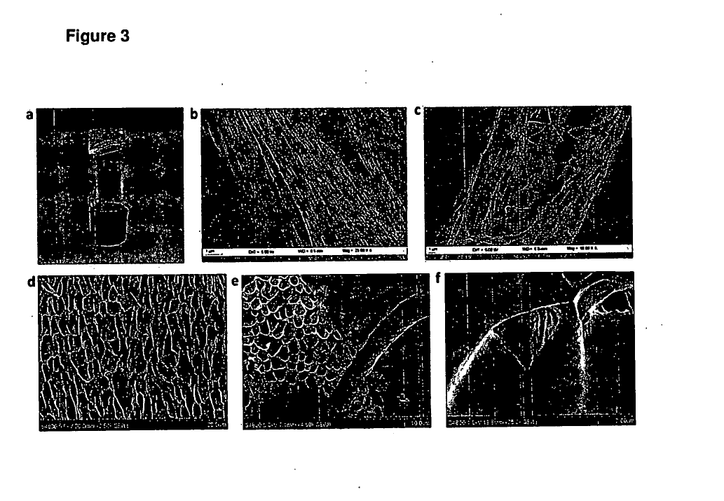

FIG. 3 shows images of a hydrogel in tris buffer. The hydrogel was prepared using CMP-KQ and cross-linked by transglutaminase. The peptide concentration was 10%.

FIG. 3a is a photograph of the CMP-KQ hydrogel in TRIS buffer;

FIGS. 3b and c are scanning electron microscopy (SEM) images of the nanofibrous assembly in the CMP-KQ hydrogel. The hydrogel was dried in different percentages of water/ethanol and diethylether followed by vacuum.

FIGS. 3d and e are Cryo-SEM images of the CMP-KQ hydrogel at different magnifications, showing the formation of honeycomb-like structure;

FIG. 3f is a Cryo-SEM image showing the presence of bundles of nanofibrous assemblies in the honeycomb structure of the CMP-KQ hydrogel;

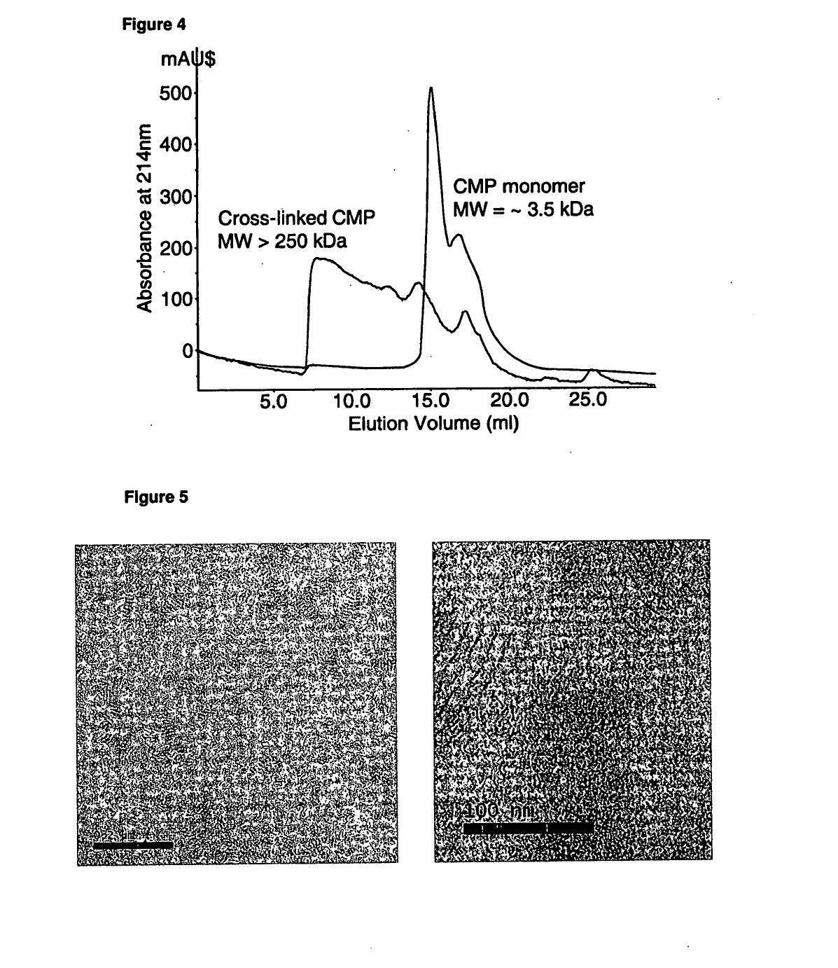

FIG. 4 is a size exclusion chromatograph of the CMP-KQ peptide monomer and a transglutaminase cross-linked assembly;

FIG. 5 shows transmission electron micrograph (TEM) images of CMP-KQ in TRIS buffer, showing the striated nanofibrous assembly structure. The concentration of CMP-KQ was 10% (w/v) and incubated at 37° C. overnight prior to measurements. The peptide solution was negatively stained with 1% Uranyl acetate solution; and

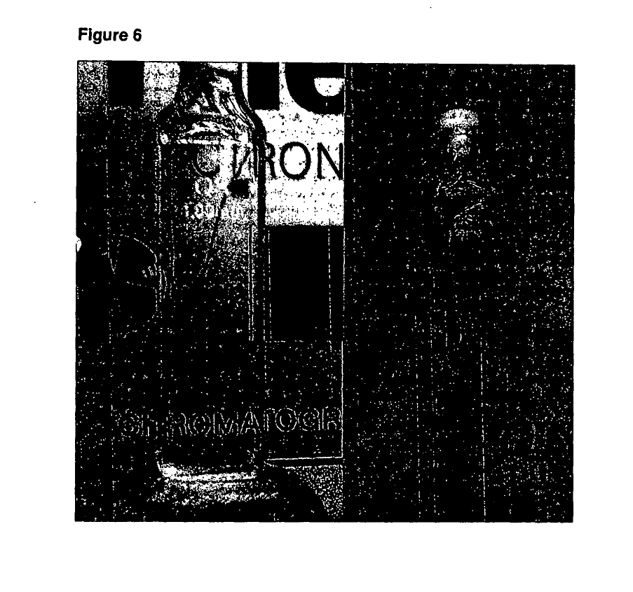

FIG. 6 shows images of the transparent hydrogels formed by enzymatically cross-linked CMP-KQ.

### EXAMPLES

### Example 1

### Synthesis of Artificial Collagen from Collagen Mimetic Peptides (CMPs)

1.1 Preparation of CMPs

Collagen mimetic peptides of the following structural formula were synthesized by standard solid phase peptide synthesis procedures:

(Gly-(A)₂-(Gly-X-Y)ₘ-M-(Gly-X-Y)ₙ-Gly-(B)₂-Gly)

wherein X and Y positions were proline and hydroxyproline respectively;

M was GFOGER;

A and B were lysine and glutamine, respectively; and

n and m were each 4.

The full peptide sequence was thus:

(SEQ ID No 1.)
Gly-Lys-Lys-(Gly-Pro-Hyp)₄-Gly-Phe-Hyp-Gly-Glu-
Arg-(Gly-Pro-Hyp)₄-Gly-Gln-Gln-Gly

Preloaded Wang resins were used as a solid support for peptide synthesis. The amino terminus of all amino acids used in this investigation was blocked by Fmoc (Fluorenylmethyloxycarbonyl) group and the side chains were protected with suitable protecting groups. Peptide synthesis grade solvents and analytical grade reagents were used for synthesis. Peptide synthesis grade N-methylpyrrolidone was employed as solvent for peptide synthesis. Each Fmoc protected amino acid was coupled on the surface of the resin sequentially by using standard solid phase protection/deprotection strategy.

Peptide synthesis was carried out in 0.1 mM scale. Typically, a calculated amount of preloaded Wang-resin was swelled overnight in N-methylpyrrolidone. After swelling, the resin was washed three times with N-methylpyrrolidone. The Fmoc protecting group on the resin was removed by treating with 20% piperidine/80% dimethylformamide (3 times). After removal of Fmoc group, the resin was washed again with N-methylpyrrolidone (3 times). A mixture of 2.5 mM of Fmoc protected amino acid and 2.5 mM of HBTU (O-(Benzotriazol-1-yl)-N,N,N′,N′-tetramethyluronium hexafluorophosphate) dissolved in N-methylpyrrolidone was added and agitated for 30 seconds using nitrogen. 5 mM of DIPEA (Diisopropylethylamine) in N-methylpyrrolidone was added and the coupling was carried out for 30 minutes under agitation by nitrogen. After coupling, the reagents were trained to waste and the resin was washed with N-methylpyrrolidone (4 times). The Fmoc group of the newly introduced amino acid was removed by treating with 20% piperidine/80% dimethylformamide (3 times). The coupling of amino acids and deprotection was carried out sequentially. After completion of the synthesis, the peptide was cleaved from the solid support using trifluoroacetic acid. The purity of the peptide was analyzed by HPLC and the molecular weight was confirmed by maldi-TOF analysis. The purity of the peptide was found to be >95% as judged by HPLC analysis.

1.2 Triple Helix Formation by CMPs

CMPs prepared as described above were dissolved in TRIS buffer and incubated at 4° C. overnight to allow spontaneous formation of a triple helix. Triple helix formation was monitored by circular dichroism (CD) spectroscopy, a technique commonly used to determine the conformation of biomaterials in solution. As shown in FIG. 1A, the CD spectrum contains a positive peak at 225 nm which is characteristic for triple helix formation.

The thermal stability of the triple helical assembly formed by the collagen mimetic peptide was monitored by circular dichroism spectroscopy (FIG. 1B) and differential scanning calorimetry (FIG. 2). The midpoint of the thermal denaturation temperature of the peptide was found to be 47° C. by circular dichroism spectroscopy and 45° C. by differential scanning calorimetry, and thus the results obtained by the two techniques were found to be in agreement with each other. These results indicate that the triple helix is stable at temperatures significantly above physiological temperatures.

1.3 Hydrogel Formation Using CMPs

10% (w/v) of the collagen mimetic peptide described above was dissolved in 50 mM TRIS and incubated overnight at 37° C. to allow triple helices and fibrils to form. The solution was then treated with 4U of transglutaminase enzyme containing 10 mM glutathione reducing agent. A reducing agent was used to reduce the disulfide bonds in the transglutaminase enzyme in order to increase the efficiency of cross-linking. Glutathione was used as the reducing agent to make the artificial collagen fully biocompatible. This mixture was incubated at 37° C. overnight to enable hydrogel formation. This process is similar to natural collagen hydrogel formation. The image of the hydrogel and its analysis by cryo-SEM are given in FIG. 3. The honeycomb structure is consistent with hydrogel formation.

As shown in FIG. 6, the collagen mimetic peptide of the invention forms a transparent hydrogel which is suitable for ophthalmic applications.

The thermal stability of the hydrogel was found to be 45° C. by tube inversion method. Briefly, a tube containing the gel was warmed to a given temperature. The tube was then turned upside down and it was observed whether the gel was ‘runny’. The maximum temperature at which the gel retained its shape i.e. did not run down the side the tube indicated the melting temperature. The hydrogel was heated to 65° C. and cooled back to room temperature by passive cooling. Hydrogel formation was observed within minutes, indicating that the collagen mimetic peptide forms thermo-reversible hydrogel, whereas native collagen is able to regain only 5-10% of the original triple helical content after heating and the remainder turns to gelatin.

In a separate experiment, 4% of the peptide (which is not a sufficient concentration to enable hydrogel formation) was treated with transglutaminase and DTT as the reducing agent. The resulting solution was analyzed by size exclusion chromatography, which revealed that the molecular weight of the cross-linked product was no more than 1M Da (FIG. 4). The solution was also analyzed by TEM by negative staining. The TEM image (FIG. 5) shows the formation of a striated assembly which is similar to natural fibrous collagens. From the length of the peptide and the spacing between the triple helices, the molecular weight of the nanofibrous assembly was deduced from the TEM striations to be roughly 1.4 MDa. This is in good agreement with the results obtained by the size exclusion chromatography.

The collagen mimetic peptides of the present invention contain all of the structural features of natural collagen, and are thus able to self-assemble into an ordered triple helix. The design of the peptides and their self-assembly behavior are easily reproducible, while their synthesis can easily be performed on an industrial scale without the need for extensive purification. The methods described herein can thus cater for the current demand for collagen for various applications. Furthermore, the experiments described herein demonstrate that it is possible to form a synthetic collagen which involves only the use of biocompatible material. The synthetic collagen of the invention thus provides a suitable alternative to natural collagen in all applications.

1. A peptide comprising the sequence Gly-(A)-(Gly-X-Y)ₘ-M-(Gly-X-Y)ₙ-Gly-(B)-Gly
wherein:
A and B are telopeptides;
X and Y are any amino acid;
m and n are integers of at least 3; and
M is a receptor binding sequence.

2. The peptide of claim 1, wherein X and/or Y is selected from proline and hydroxyproline.

3. The peptide of claim 2, wherein X is proline and Y is hydroxyproline.

4. The peptide of claim 1, wherein each of A and B is a sequence of two or more amino acids, the sequence comprising at least one lysine or glutamine residue.

5. The peptide of claim 4, wherein A comprises or consists of the sequence Lys-Lys.

6. The peptide of claim 4, wherein B comprises or consists of the sequence Gin-Gin.

7. The peptide of claim 1, wherein M is an integrin binding sequence.

8. The peptide of claim 7, wherein M comprises or consists of the sequence GFOGER, GFOGDR or GFOLDV.

9. A method for preparing a hydrogel, the method comprising:
providing a solution of the peptide of claim 1;
allowing the peptides to assemble into higher order structures; and
cross-linking the peptides.

10. The method of claim 9, wherein cross-linking is effected by adding an enzyme to the solution.

11. The method of claim 10, wherein the enzyme is lysyl oxidase or transglutaminase.

12. The method of claim 9, further comprising adding cells to the higher order structures formed from the peptide, and effecting cross-linking in the presence of the cells.

13. A hydrogel comprising the peptide according to claim 1.

14. A hydrogel obtainable by the method according to claim 9.

15. The hydrogel of claim 13, wherein at least 50% of the hydrogel is constituted by the peptide of claim 1.

16. The hydrogel of claim 13, wherein the hydrogel has a thermal stability of from 40 to 50° C.

17. The hydrogel of claim 13, wherein the hydrogel is transparent.

18. A material comprising the hydrogel of claim 13.

19. An article comprising the material of claim 18.

20. The article of claim 19, wherein the article is a wound dressing, a hemostatic sponge, a healing aid or a corneal shield.

21. (canceled)

22. An artificial tissue comprising the hydrogel of claim 13.

23. The artificial tissue of claim 22, which is an artificial tendon, organ, ligament, cornea, skin, cartilage, blood vessel, bone graft or heart valve.

24-25. (canceled)

26. A method of treating diseased or damaged tissue, the method comprising administering the hydrogel of claim 13 to a patient in need thereof.
